(19) United States
(12) Patent Application Publication
(10) Pub. No.: US 2023/0203749 A1
(43) Pub. Date: Jun. 29, 2023

(54) ANTIVIRAL FABRIC

(71) Applicant: AMOGREENTECH CO., LTD., Gimpo-si, Gyeonggi-do (KR)

(72) Inventors: Seon Ho JANG, Gimpo-si, Gyeonggi-do (KR); Dong Sik SEO, Gimpo-si, Gyeonggi-do (KR); In Yong SEO, Gimpo-si, Gyeonggi-do (KR); Chan KIM, Gimpo-si, Gyeonggi-do (KR); Seoung Hoon LEE, Gimpo-si, Gyeonggi-do (KR)

(73) Assignee: AMOGREENTECH CO., LTD., Gimpo-si, Gyeonggi-do (KR)

(21) Appl. No.: 17/999,465

(22) PCT Filed: May 24, 2021
(86) PCT No.: PCT/KR2021/006419

§ 371 Date: Nov. 21, 2022

(30) Foreign Application Priority Data
May 22, 2020 (KR) 10-2020-0061555
May 22, 2020 (KR) 10-2020-0061556

(51) Int. Cl. D06M 16/00; C07K 14/00; A61L 9/16; C07K 7/04; B82Y 30/00; C09D 189/00

(52) U.S. Cl. CPC D06M 16/003; C07K 14/001; A61L 9/16; C07K 7/04; B82Y 30/00; C09D 189/00; C07K 2319/00; D10B 2401/13; D06M 2200/12

(57) ABSTRACT

An antiviral fabric including a fabric and an antiviral coating layer which is provided in the fabric and which includes an antiviral fusion protein having an antiviral motif bound to an adhesive protein. The antiviral fabric has excellent processability that enables simple implementation of the antiviral coating layer even on the curved surface of a fiber, the porous surface of a fabric, or a recessed or protruding surface. In addition, the antiviral fabric can have activity persistence that enable maintenance of antiviral activity for a long time without losing the same according to a condition during preparation, storage, use and washing, while having adhesion persistence that enables the maintenance of an adhesive state for a long time after the antiviral coating layer is formed on the surface thereof, and thus can be widely applied to various articles for which fabric is used.

### CROSS-REFERENCE TO RELATED APPLICATIONS

This application is a 35 U.S.C. 371 National Phase Entry application from PCT/KR2021/006419 filed May 24, 2021, which claims priority to and the benefit of Korean Patent Application Nos. 10-2020-0061555 and 10-2020-0061556, both filed on May 22, 2020, the disclosures of which are incorporated herein by reference in their entirety.

### TECHNICAL FIELD

The present invention relates to a fabric, and more particularly, to a fabric having antiviral activity.

### BACKGROUND

SARS, which began to spread in early 2003, MERS, which began infecting people in 2012 and also caused 186 cases in South Korea in 2015, and COVID-19 that began in late 2019 and has spread all over the world to date are all caused by viruses, and the need for a sanitary and safe living environment has significantly increased against this background. In particular, considering that it takes a considerable amount of time to develop a vaccine after the outbreak of diseases caused by viruses, the demand for various antiviral products to create a safe living environment is further increasing.

All of the above-described diseases are caused by coronaviruses, and ethanol, sodium hypochlorite, an iodophor, peracetic acid, formaldehyde, glutaraldehyde, and ethylene oxide gas are reported to be effective as disinfectants against coronaviruses. Further, 1-adamantanamine hydrochloride, thiosemicarbazide, an arabinosyl nucleoside, nucleoside, 2,3-dideoxynucleoside, pyrophosphate derivatives and the like are known as antiviral agents.

However, since the activity of components having these antiviral characteristics is temporary or the activity may be easily lost due to various external factors such as temperature and humidity, the antiviral effect and durability of antiviral fabrics cannot be expected.

In addition, even when there is an antiviral effect by itself, antiviral activity is lost during the process of applying the components to fabrics or there is also a problem in that a process of providing fabrics with antiviral components is not easy.

Furthermore, even though the antiviral component is provided on the fabric while maintaining antiviral activity, there is also a problem in durability because the antiviral component may be easily detached from the fabric through rubbing, washing, and the like.

### SUMMARY OF THE INVENTION

The present invention has been devised in consideration of the above points, and an object of the present invention is to provide an antiviral fabric having excellent processability enabling easy provision on the surface of a fiber or fabric, having adhesion sustainability enabling an adhesive state to be maintained for a long period of time after being adhered to a surface, and having activity sustainability enabling antiviral activity to be maintained for a long period of time without a loss in activity according to external conditions during preparation, storage, use and washing.

Further, another object of the present invention is to provide an antiviral fabric for medical and quarantine uses designed to have a moisture-permeable waterproof function such that liquids such as saliva and blood of patients containing viruses are impermeable while the sweat of workers wearing clothes made of an antiviral fabric is smoothly discharged.

To solve the above-described problems, the present invention provides an antiviral fabric including a fabric, and an antiviral coating layer provided on the fabric and including an antiviral fusion protein in which an antiviral motif is bound to an adhesive protein.

According to one embodiment of the present invention, the antiviral motif may target a protein that binds to a host cell receptor to disable or disrupt the protein, or may perform the function of disrupting the viral membrane.

Furthermore, the adhesive protein may be a mussel-derived adhesive protein.

Further, the antiviral motif is any one peptide selected from the group consisting of amino acid sequences of SEQ ID NOS: 1 to 8, or a peptide in which one or more amino acid sequences selected from the above group are linked, and the adhesive protein may be any one protein selected from the group consisting of amino acid sequences of SEQ ID NOS: 9 to 22, or a protein in which one or more amino acid sequences selected from the above group are linked.

In addition, the antiviral coating layer may be formed on a fiber by aggregating particles formed of the antiviral fusion protein.

Furthermore, the antiviral coating layer may be formed through an antiviral coating composition including an antiviral fusion protein and an aggregation-inducing component including a carbodiimide-based coupling agent and a hydroxy succinimide-based reactive agent.

Further, the adhesive protein contains a DOPA residue, and the antiviral fusion protein may be immobilized on a fiber through the DOPA residue. In this case, the DOPA residue may be a DOPA residue into which some or all tyrosine residues of the adhesive protein are modified through an enzyme.

In addition, the fabric may include any one or more of a woven fabric, a knitted fabric and a non-woven fabric.

Furthermore, the fabric may be composed of a single layer such that at least a part thereof has a moisture-permeable waterproof function, or may be composed of multiple layers including at least one moisture-permeable waterproof layer.

Further, the moisture-permeable waterproof layer may be a nanofiber web provided with a fiber-forming component including a fluorine-based compound and any one or more additives selected from a water repellent, an oil repellent, and a moisture absorbent.

In addition, the fabric may include a first yarn having an antiviral coating layer formed on the surface and a second yarn containing silver.

Furthermore, the first yarn and the second yarn may constitute a single-layered fabric, or a first fabric layer including the first yarn and a second fabric layer including the second yarn may be laminated to constitute a fabric.

Further, the present invention provides clothing, a sanitary product, bedding, or an article for clothing and quarantine uses, which includes the antiviral fabric according to the present invention.

The antiviral fabric according to the present invention has excellent processability that enables simple implementation of an antiviral coating layer even on the curved surface of a fiber, the porous surface of a fabric, or a recessed or protruding surface. In addition, the present invention can have activity persistence that enables the maintenance of antiviral activity for a long time without losing the same according to conditions during preparation, storage, use and washing, while having adhesion persistence that enables the maintenance of an adhesive state for a long time after the antiviral coating layer is formed on the surface thereof, and thus can be widely applied to a sanitary product such as various tissues and diapers, regular clothes such as underwear, socks, hats, gloves, tops and bottoms, various types of bedding such as pillowcases and comforters, fillers used for clothing, bedding, and the like, and articles for medical and quarantine uses.

### BRIEF DESCRIPTION OF THE DRAWINGS

FIG. 1 is a cross-sectional schematic view of a coronavirus.

FIG. 2 is a schematic view related to targets by which antiviral motifs can act against coronaviruses.

### DETAILED DESCRIPTION

Hereinafter, embodiments of the present invention will be described in detail with reference to the accompanying drawings such that a person with ordinary skill in the art to which the present invention pertains can easily carry out the present invention. The present invention can be embodied in various forms, and is not limited to the embodiments described herein.

An antiviral fabric according to one embodiment of the present invention is provided with a fabric and an antiviral coating layer which includes an antiviral component provided in the fabric.

The fabric refers to various textile products implemented through fibers, and may include, for example, any one or more of a woven fabric, a knitted fabric, and a non-woven fabric.

The woven and knitted fabrics refer to members having a tendency to arrange yarns, and the non-woven fabric refers to a member in which yarns are randomly arranged.

First, the structure of the woven fabric may be formed by any method selected from the group consisting of a plain weave, a twill weave, a satin weave and a double weave. When the plain weave, the twill weave and the satin weave are said to have a ternary structure, a specific method for weaving each of the ternary structures is performed by a typical weaving method, these weaves may be woven fabrics whose structures are modified, or changed by blending several structures, and for example, examples of a fancy plain weave include a ribbed plain weave, a basket weave, and the like, examples of a fancy twill weave include an elongated twill weave, a broken twill weave, a skip twill weave, a pointed twill weave, and the like, and examples of a fancy satin weave include an irregular satin weave, a double satin weave, an extended satin weave, a granite satin weave, and the like. The double weave is a woven fabric in which either one of the warp yarn or weft yarn is doubled or both are doubled, and a specific method for the weaving method may be a typical method of weaving a double weave. In addition, the warp yarn and weft yarn densities of the woven fabric are not particularly limited. Accordingly, it should be noted that a mesh form formed with very low warp yarn and weft yarn densities can be seen as an example of a woven fabric.

Further, the knitting may be performed by a method of weft knitting or warp knitting, and a specific method of weft knitting and warp knitting may be a typical method of weft knitting or warp knitting.

Weft knitting products such as flat pieces, rubber pieces, and pearl pieces may be specifically manufactured by the weft knitting, and warp knitting products such as tricot, milanese, and raschel can be specifically manufactured by the warp knitting.

In addition, since the non-woven fabric is arranged such that the fibers are not oriented vertically and horizontally, a known non-woven fabric may be used, and the manufacturing method thereof is not limited. For example, the non-woven may be a chemically-bonded non-woven fabric, a thermally-bonded non-woven fabric, an air-laid non-woven fabric, a needle-punched non-woven fabric, a spunbond non-woven fabric, a melt-blown non-woven fabric, a stitch-bonded non-woven fabric, and/or an electrospun nanofiber web.

The fabric may be formed by laminating one of the above-mentioned woven fabric, knitted fabric and non-woven fabric in a single layer or multiple layers, or by laminating two or more of these in multiple layers, and the lamination order of these layers may be appropriately changed depending on the purpose.

Furthermore, the fabric may have a moisture-permeable waterproof function so as to prevent the permeation of virus-containing liquids, for example, a patient's saliva and blood, from the outside while the sweat of workers wearing clothes implemented by the fabric can be easily discharged.

The moisture-permeable waterproof function may allow at least a part of the fabric to have a moisture-permeable waterproof function when the fabric is composed of a single layer, or allow any one or more layers of the fabric to become a moisture-permeable waterproof layer when the fabric is composed of multiple layers.

First, when the fabric is composed of a single layer, the moisture-permeable waterproof function may be provided on one surface of the fabric based on the thickness direction of the fabric, or the moisture-permeable waterproof function may be provided throughout the thickness direction. Meanwhile, the moisture-permeable waterproof function may be achieved by adding one or more additives such as a water repellent, an oil repellent, and a moisture absorbent, for example, to reduce permeability to water. Further, it is possible to reduce or prevent the permeability to water by constituting a hydrophobic material as a material for fibers constituting the fabric or adjusting the diameter, basis weight, density, porosity, and pore diameter of the fibers.

In addition, when the fabric is composed of multiple layers, the fabric may include at least one layer with a moisture-permeable waterproof function. Such a moisture-permeable waterproof layer may be a known porous member having a moisture-permeable waterproof function. For example, the moisture-permeable waterproof layer may be a nanofiber web including a fiber-forming component including a fluorine-based compound and any one or more additives selected from a water repellent, an oil repellent, and a moisture absorbent.

When the nanofiber web having the moisture-permeable waterproof function is specifically described, the fiber-forming component of the nanofibers constituting the nanofiber web may be a polymer compound having a hydrophobic property, and may be, for example, a fluorine-based polymer compound, in order to make moisture in the air impermeable. Specifically, the fluorine-based compound may include one or more compounds selected from the group consisting of polytetrafluoroethylene (PTFE)-based, tetrafluoroethylene-perfluoroalkyl vinyl ether copolymer (PFA)-based, tetrafluoroethylene-hexafluoropropylene copolymer (FEP)-based, tetrafluoroethylene-hexafluoropropylene-perfluoroalkyl vinyl ether copolymer (EPE)-based, tetrafluoroethylene-ethylene copolymer (ETFE)-based, polychlorotrifluoroethylene (PCTFE)-based, chlorotrifluoroethylene-ethylene copolymer (ECTFE)-based and polyvinylidene fluoride (PVDF)-based compounds. However, some or all of the fiber-forming components of the nanofibers may not be fluorine-based compounds, considering that permeability to water can be reduced by additives to be described below. In this case, as the fiber-forming component, any material known to be suitable for manufacturing nanofibers can be used without limitation, and non-limiting examples thereof may be a polyethylene glycol derivative including polyethylene glycol dialkyl ether and polyethylene glycol dialkyl ester, a polyoxide including poly(oxymethylene-oligo-oxyethylene), polyethylene oxide and polypropylene oxide, polyvinyl acetate, poly(vinylpyrrolidone-vinyl acetate), polystyrene and polystyrene acrylonitrile copolymers, polyacrylonitrile (PAN), a polyacrylonitrile copolymer including a polyacrylonitrile methylmethacrylate copolymer, polymethyl methacrylate, polymethyl methacrylate copolymer or a mixture thereof.

Meanwhile, the nanofiber may include one or more additives such as a water repellent, an oil repellent, and a moisture absorbent to reduce permeability to water. When the nanofiber is manufactured, the additive may be mixed with the fiber-forming component to be provided in the nanofiber, or the additive may be provided by treating the outer surface or the outer surface and the inner surface of the fiber web manufactured by including the nanofiber with the additive, and in this case, the treatment may be a typical coating method, so that a detailed description thereof will be omitted.

In this case, the water repellent is not particularly limited, but a silicone resin, a fluorine resin, paraffin wax, an alkyl ketene dimer and the like may be applied. In particular, since a fluorine-based water repellent can impart not only water repellency but also oil repellency, the fluorine-based water repellent is very advantageous in imparting higher water repellency or oil repellency.

The oil repellent may be a fluorine-based or silicone-based component, but is not limited thereto.

The moisture absorbent may be a known component capable of removing water or moisture, and may be, for example, a metal powder such as alumina, a water reactive moisture absorbent such as a metal oxide, a metal salt, or phosphorus pentoxide (P₂O₅), or a physical moisture absorbent such as silica, zeolite, titania, zirconia or montmorillonite.

In addition, the nanofibers may have an average diameter of 0.1 to 2 μm. Furthermore, the nanofiber web may have a thickness of 3 to 40 μm depending on the required water pressure resistance. Further, the nanofiber web may have a pore diameter of 5 μm or less and a porosity of 20 to 90%. In addition, the nanofiber web may have a wetting angle of 110° or more on the surface where the inflowing air is first brought into contact, and through this, it is possible to easily inhibit the passage of inflowing moisture and remove the inflowing moisture.

Furthermore, an antiviral coating layer provided with an antiviral component is provided on the above-described fabric.

The antiviral coating layer contains an antiviral coating layer in the entire region in the thickness direction over the surface and rear surface of the fabric, or an antiviral coating layer is not provided on the surface or rear surface of the fabric, or in the center in the thickness direction of the fabric, and an antiviral coating layer may be provided only on the surface portion and rear surface portion of the fabric. Further, when the fabric is composed of multiple layers, the antiviral coating layer may be provided only on some of the layers or provided over the entire layer.

In addition, since the fabric is typically a member having a predetermined degree of ventilation, pores of the fabric located in a part where the antiviral coating layer is formed may be provided such that the pores are completely blocked by the antiviral coating layer or only some of the pores are blocked. Alternatively, pores of the fabric located in a part where the antiviral coating layer is formed may be provided so as not to be completely blocked.

The antiviral component provided in the antiviral coating layer includes an antiviral fusion protein, and the antiviral fusion protein is formed by allowing an antiviral motif to bind to an adhesive protein.

The antiviral motif may be a motif that functions to suppress viral proliferation, destroy the virus itself, or block infection by participating in a mechanism by which a host is infected by the virus.

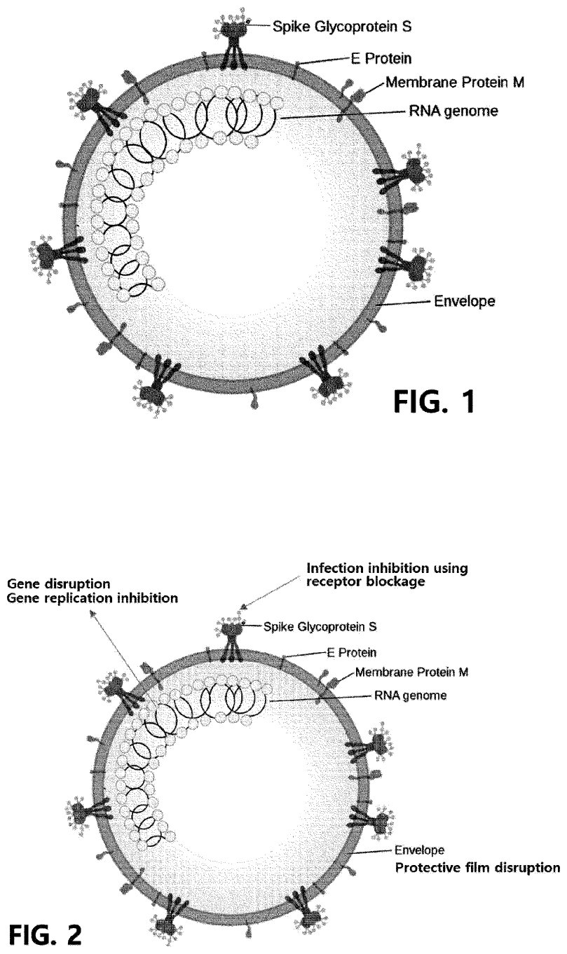

For example, referring to FIGS. 1 and 2, the antiviral motif may have a function of directly or indirectly disrupting the outer membrane, which is the protective film of the virus. Alternatively, when a virus infects a host cell, the antiviral motif may have a function of directly or indirectly disrupting a protein (for example, a spike protein of a coronavirus) bound to the host cell, or directly or indirectly disabling the protein. Here, the direct or indirect means that the antiviral motif directly performs the corresponding function or participates in the initial or intermediate process in ultimately performing the corresponding function.

The antiviral motif can be used without limitation as long as the antiviral motif is a known motif that is known to have an antiviral effect such as destruction or inactivation of the above-described virus. For example, the antiviral motif may be any one peptide selected from the group consisting of amino acid sequences of SEQ ID NOS: 1 to 8, a peptide in which one or more amino acid sequences selected from the above group are linked, or a peptide including one or more amino acid sequences selected from the above group as a basic sequence. For example, the motifs according to SEQ ID NOS: 1 and 2 may be useful for SARS coronaviruses, the motifs according to SEQ ID NOS: 3 to 8 may be useful for influenza A viruses, and in addition, the motif according to SEQ ID NO: 7 may also be useful for HSV.

Further, the antiviral motif may be, for example, a peptide with 3 to 100 amino acids, more preferably 3 to 20 amino acids.

In addition, the virus which the antiviral motif targets is not limited as long as the virus is a known virus, and non-limiting examples thereof include JV, HSV, HIV, IPNV, VHSV, SHRV, HCMV, IAV, Japanese encephalitis virus, Ebola virus, rhinovirus, adenovirus, measles virus, hepatitis B virus, influenza A, and the like.

The above-described antiviral motif itself may be included in the coating composition to treat the surface of the fabric or yarn constituting the fabric, but it is not easy to immobilize the antiviral motif alone on the surface of the fabric or yarn constituting the fabric for a long time. Accordingly, the present invention is implemented in the form of a fusion protein in which the antiviral motif is bound to a conjugated protein. The adhesive proteins may function as an adhesive component that provides adhesion between the antiviral motif and the surface of the fabric or yarn constituting the fabric. Meanwhile, when protein is used as an adhesive component, there is an advantage in that the coating composition can be used for the use of a sanitary product, clothing, a mask, a hat, gloves, and the like capable of directly affecting the human body because the protein is non-toxic compared to a polymer-based adhesive component.

Furthermore, the bond between the antiviral motif and the adhesive protein may be a covalent bond, and more specifically, the antiviral motif may be bound to the carboxy terminus, the amino terminus, or both the carboxy terminus and the amino terminus of the adhesive protein by a peptide bond. Meanwhile, the antiviral motif and the adhesive protein may be bound by a known method, and for example, may be prepared by a recombinant protein production method using Escherichia coli. Meanwhile, the adhesive protein and the antiviral motif may be directly bound by a covalent bond, but the bond is not limited thereto, and it should be noted that the adhesive protein and the antiviral motif may be indirectly bound by a covalent bond, and the like by adding a third material as a spacer.

Further, the adhesive protein can be used without limitation as long as the adhesive protein is a protein having a known adhesion function, but may be a mussel-derived adhesive protein, and a known adhesive protein commonly called a mussel-derived adhesive protein can be used without limitation. Preferably, the adhesive protein may include any one protein selected from the group consisting of amino acid sequences of SEQ ID NOS: 9 to 22 or a protein in which one or more amino acid sequences selected from the above group are linked.

In addition, the antiviral component may further include a heterologous material having antiviral functions in addition to the above-described antiviral fusion protein. The heterologous material may be a known organic material or inorganic material. For example, the heterologous material may be an inorganic material with a substituent having proton-donating or proton-accepting properties disposed on the surface with which the virus is brought into contact, and specific examples thereof include phosphate compounds of titanium group elements such as zirconium phosphate, hafnium phosphate, and titanium phosphate and inorganic phosphate compounds such as aluminum phosphate and hydroxyapatite (phosphate minerals); inorganic silicate compounds such as magnesium silicate, silica gel, aluminosilicate, sepiolite (hydrous magnesium silicate), montmorillonite (silicate mineral), and zeolite (aluminosilicate); alumina, titania, hydrous titanium oxide, and the like. Alternatively, the heterologous material may be a metal such as silver or a salt containing ions thereof.

Furthermore, the antiviral coating composition may further include a solvent which dissolves the above-described antiviral fusion protein or a buffer solution which stabilizes the above-described antiviral fusion protein. The solvent may be water and/or an organic solvent, and 20 to 100 mM Tris or a sodium bicarbonate buffer solution with a pH of 8 to 8.5 may be used.

Further, the above-described antiviral fusion protein may be contained at a concentration of 0.001 to 1 mg/ml, and as another example, 0.001 to 0.2 mg/ml in the antiviral coating composition, and when the above-described antiviral fusion protein is contained at a high concentration, antiviral properties may be improved, but there is a concern that it is possible to clog the porous structure of the member.

Meanwhile, among antiviral fusion proteins, an adhesive protein, particularly, a mussel-derived adhesive protein is known to have adhesion properties itself, but as a result of studies by the present inventors, when these adhesive proteins are used as they are, they exhibit no or insignificant levels of adhesion (or cohesion) properties, making it difficult to immobilize the antiviral motif on the surface of the fabric or yarn constituting the fabric. Accordingly, the antiviral fusion protein in the antiviral coating composition, particularly, the adhesive protein among the antiviral fusion proteins, may contain a DOPA residue in order to exhibit more improved adhesion properties with the surface of the fabric or yarn constituting the fabric. Alternatively, the antiviral coating composition may further contain an aggregation-inducing component including a carbodiimide-based coupling agent and a hydroxy succinimide-based reactive agent.

First, an antiviral coating composition in which the adhesive protein contains a DOPA residue in the antiviral fusion protein will be described.

As described above, in the case of an adhesive protein, particularly, a mussel-derived adhesive protein by itself, it is difficult for the adhesive protein itself to exhibit sufficient adhesive and cohesive properties. However, when a DOPA residue is contained, there is an advantage in that the antiviral motif can be easily and strongly immobilized on the surface of the fabric or yarn constituting the fabric through the DOPA residue. The DOPA residue may be provided in an antiviral fusion protein through modification, the modification modifies some or all of the tyrosine residues contained within the adhesive protein into DOPA residues, and such modification may be performed appropriately using known methods. For example, the modification may be performed using an enzyme, and the enzyme may be, for example, tyrosinase.

Specifically, the modification may be performed by including (1) preparing a solution in which an antiviral fusion protein is dissolved in a buffer solution containing ascorbic acid, (2) preparing the solution in an oxygen-saturated state, and then modifying a tyrosine residue in an adhesive protein into a DOPA residue by mixing tyrosinase with the solution and (3) performing desalting with acetic acid. In this case, in Step (1), the buffer solution contains ascorbic acid as an antioxidant at a concentration of 25 to 100 mM, an antiviral fusion protein may be provided at a final concentration of 0.1 to 10 mg/ml in the solution, and the buffer solution may include 20 to 100 mM sodium acetate and 20 to 100 mM sodium borate.

In addition, Step (2) may be performed by saturating the prepared solution with oxygen in the solution while injecting oxygen for 10 minutes to 1 hour, adding tyrosinase to a final concentration of 10 to 50 μg/ml, mixing and stirring the resulting mixture under oxygen conditions for 30 minutes to 2 hours, and then terminating the reaction by adding acetic acid to a final concentration of 2 to 10%.

Furthermore, Step (3) may be performed by desalting and concentrating the reaction solution in which the reaction is terminated with a 1 to 10% acetic acid solution.

Also, after the modification is performed up to Step (3), the antiviral fusion protein modified to contain a DOPA residue may be prepared in a powder form by lyophilization.

An antiviral fusion protein containing a DOPA residue prepared by the above-described method may be easily immobilized on the desired surface of the fabric or yarn constituting the fabric without additional adhesive components, and as other adhesive components are not used, there is an advantage in that it is possible to prevent the deterioration or inactivation of activity due to unintended chemical reactions between other components and antiviral motifs and physical blocking.

However, as the improvement of the adhesion of the antiviral fusion protein by modification into the above-described DOPA residue requires additional cost, time, and effort, the coating composition according to one embodiment of the present invention may further include an aggregation-inducing component including a carbodiimide-based coupling agent and a hydroxy succinimide-based reactive agent. The aggregation-inducing component is a material that introduces an antiviral fusion protein to the surface of the fabric or yarn constituting the fabric, and may improve adhesion between the coating layer of the antiviral fusion protein and the surface of the target compared to the case where the surface of the fabric or yarn constituting the fabric is treated with the antiviral fusion protein alone using a typical method. Specifically, the aggregation-inducing component aggregates the antiviral fusion protein into particles, and an antiviral coating layer may be implemented in such a manner that these particles are adsorbed on the surface of the fabric or yarn constituting the fabric to form aggregates. An antiviral coating composition including an aggregation-inducing component may immobilize an antiviral motif on the surface of a target with improved adhesive strength, may also sustain antiviral performance by preventing or minimizing degradation, denaturation, and the like of the antiviral motif at room temperature for a long period of time, and may improve storage stability.

Meanwhile, it is difficult to see that the granular form in which the antiviral fusion protein is aggregated by the aggregation-inducing component is due to a specific chemical bond between the fusion proteins, for example, an amino bond between a carboxyl group and an amine group by a carbodiimide-based coupling agent known in the art, which is because a plurality of hydroxy groups included in an adhesive protein, for example, a mussel-derived adhesive protein may also react with a carbodiimide-based coupling agent. Therefore, it is difficult to see that the granular form formed by the antiviral fusion protein having a plurality of reaction sites according to the present invention is due to a specific reaction and the resulting chemical bond, and it may be seen as a unique result occurring according to the combination between antiviral fusion proteins containing an aggregation-inducing component and an adhesive protein.

The carbodiimide-based coupling agent can be used without limitation in the case of a coupling agent that allows antiviral fusion proteins to bind to each other, and may be, for example, 1-[3-(dimethylamino)propyl]-3-ethylcarboimide hydrochloride (EDC) or N,N′-dicyclohexylcarbodiimide (DCC).

Furthermore, the hydroxy succinimide-based reactive agent is provided to increase the efficiency with which antiviral fusion proteins are aggregated with each other by preventing the antiviral fusion protein coupled with carbodiimide from being hydrated, and may be, for example, one of N-hydroxysuccinimide (NHS) and N-hydroxysulfosuccinimide (Sulfo-NHS) or a mixture thereof.

The aggregation-inducing component may include the carbodiimide-based coupling agent and the hydroxy succinimide-based reactive agent at a weight ratio of 1:0.5 to 20, more preferably 1:0.5 to 10, and even more preferably 1:0.5 to 3. When they are not included at an appropriate ratio, it is difficult to achieve the intended effect of the present invention, and there is a concern that when the durability and storage periods of the implemented antiviral coating layer are extended, the activity of the antiviral motif deteriorates.

Further, the aggregation-inducing component may further include sodium acetate, a phosphate buffer solution, or an MES buffer solution as an active component to improve reactivity. In this case, the active component may be included in an amount of 20 to 50 parts by weight with respect to 100 parts by weight of the total weight of the carbodiimide-based coupling agent and the hydroxy succinimide-based reactive agent, and this may be more advantageous in achieving the object of the present invention.

Meanwhile, the above-described aggregation-inducing component may be added to the coating composition as a liquid phase dissolved in a solvent, and in this case, water or an organic solvent may be used as the solvent, and preferably, water may be used, and ethanol may be further included as a solvent in terms of increasing the volatilization rate of the solvent in the coating composition. When the evaporation of the solvent is delayed after the treatment of the coating composition, the antiviral coating composition may flow from the fabric or yarn constituting the fabric, so that there is a concern that it may be difficult to form an antiviral coating layer with desired content and thickness and pores in the porous structure may be clogged.

Meanwhile, when the antiviral coating composition further includes an aggregation-inducing component, the reaction between the antiviral fusion protein and the aggregation-inducing component may continue to occur in an antiviral coating composition state before treatment of the surface of the fabric or yarn constituting the fabric with the composition due to the aggregation-inducing component, and when the fabric or yarn constituting the fabric is treated with the antiviral coating composition after the above reaction has excessively progressed beyond the target level, it is difficult to form an antiviral coating layer, or even though the antiviral coating layer is formed, adhesive strength is weak, or the antiviral coating layer is formed to have a rough coating surface, or it is not easy to coat the antiviral coating composition, such as a portion which is not coated is present, and the quality of a prepared antiviral coating layer may be poor. In addition, it may be difficult for the antiviral motif in the antiviral coating layer to be exposed to the outside, so that there is a concern that antiviral characteristics deteriorate. In addition, there is a concern that pores in the porous structure may be clogged.

Accordingly, it is preferred that the antiviral coating composition further includes a delaying component capable of delaying the reaction between the antiviral fusion protein and the aggregation-inducing component, or the coating composition is stored under conditions capable of delaying the reaction, as an example, under a low-temperature condition which is 0 to 15° C., as another example, 0 to 10° C.

Alternatively, as another exemplary embodiment, the antiviral coating composition is prepared using a first solution including an antiviral component and a second solution including an aggregation-inducing component, and then the first solution and the second solution are mixed according to the timing of treatment of the surface of the fabric or yarn constituting the fabric, and then the surface of the fabric or yarn constituting the fabric may be immediately treated with the mixture, or the surface of the fabric or yarn constituting the fabric may be treated with the mixture after imparting a predetermined reaction time after mixing of the components.

When the process of preparing the coating composition containing the above-described aggregation-inducing component is looked at in detail, a second solution in which the aggregation-inducing component including the carbodiimide-based coupling agent and the reactive agent is dissolved in a solvent and a first solution in which the antiviral fusion protein is dissolved are each prepared, and then these solutions may be mixed at a predetermined content.

The first solution may be prepared by dissolving the prepared antiviral fusion protein in a solvent, for example, water.

In addition, the second solution may be prepared by mixing a carbodiimide-based coupling agent, a hydroxy succinimide-based reactive agent and a solvent, for example, water and/or ethanol, or prepared by preparing each of a mixed solution of a carbodiimide-based coupling agent and a solvent and a mixed solution of a hydroxy succinimide-based reactive agent and a solvent, and then mixing these mixed solutions.

In this case, the above-described active component may be included in the second solution, and for example, the second solution may be an activated solution obtained by mixing a carbodiimide-based coupling agent, a hydroxy succinimide-based reactive agent, and the active component, and then reacting the mixture for 1 to 60 minutes. Alternatively, the second solution may also be prepared by preparing each of a first mixed solution of a carbodiimide-based coupling agent and an active component and a second mixed solution of a hydroxy succinimide-based reactive agent and an active component, and then mixing these mixed solutions. In this case, two mixed solutions may be mixed with the first solution immediately after being mixed, but as another example, the two mixed solutions are mixed, and then the second solution may be prepared by inducing a reaction for 30 to 60 minutes.

Next, a step of mixing the prepared first and second solutions may be performed. In this case, the mixing ratio of the first solution and the second solution may be appropriately changed in consideration of a specific method of treating the surface with the antiviral coating composition, the thickness of a coating layer to be formed, the degree of antiviral activity, and the like. As an example, the first solution and the second solution may be mixed by adjusting the content such that the total weight of the carbodiimide-based coupling agent and the hydroxy succinimide-based reactive agent is 50 to 200 parts by weight, as another example, 80 to 120 parts by weight, with respect to 100 parts by weight of the antiviral fusion protein. When the total weight of the carbodiimide-based coupling agent and the hydroxy succinimide-based reactive agent is less than 50 parts by weight, it may be difficult to implement a granular form, so that the coatability on the surface of the fabric or yarn constituting the fabric may deteriorate. Furthermore, when the total content exceeds 200 parts by weight, the coating layer may be peeled off.

Meanwhile, after the mixing of the first solution and the second solution prepared as described above is performed, an aging step of inducing a reaction between the antiviral fusion protein in the first solution and the aggregation-inducing component in the second solution may be further performed.

Here, inducing a predetermined reaction means that the antiviral fusion protein is initiated to aggregate on the antiviral coating composition, or the surface of the fabric or yarn constituting the fabric is treated in a state in which the antiviral fusion protein has already been aggregated to form particles having a predetermined size, and is not limited thereto, and it should be noted that an aggregation reaction may be induced by treating the surface of the fabric or yarn constituting the fabric with the antiviral coating composition immediately after mixing the solutions. The aging step may be controlled by the content of the fusion protein and the aggregation-inducing component, the method of treating the target with the antiviral coating composition, and the like, and may be performed, as an example, for more than 0 to 300 minutes, and as another example, for 30 to 60 minutes. The aging time may also vary depending on the temperature conditions during aging, and the aging time during aging at low temperature may be increased. For example, when the surface of the fabric or yarn constituting the fabric is coated with the antiviral coating composition using an impregnation method at room temperature, for example, 20 to 25° C., the aging time may be, as an example, 10 minutes or more, and as another example, 30 minutes, or 40 minutes or more. As another example, when the antiviral coating composition is electrosprayed, the aging time may be, as an example, within 10 minutes, and as another example, within 8 minutes, 6 minutes, 4 minutes, and 2 minutes, for example, at 20 to 25° C.

In addition to the components described above, the above-described antiviral coating composition may further contain a component such as a dispersant, a leveling agent, a viscosity modifier, and an antifoaming agent, which are contained in a typical coating composition, and the specific types and contents thereof are not particularly limited in the present invention, because the known types may be used by adjusting the content to an appropriate content depending on the purpose.

Further, the antiviral fusion protein in the above-described antiviral coating composition may be immobilized on the surface of the fabric or yarn constituting the fabric through the treating of the surface of the fabric or yarn constituting the fabric with the antiviral coating composition and the drying of the antiviral coating composition. Specifically, since the fabric to be treated with the antiviral coating composition is typically a member having a predetermined degree of ventilation, the fabric may be treated with the antiviral coating composition such that all of the pores contained in the fabric or some of the pores are blocked, or may be treated with the antiviral coating composition such that all the pores are not blocked. In this case, when the fabric is directly treated with the antiviral coating composition, the antiviral coating layer may be formed such that the pores of the fabric are not blocked by appropriately adjusting the concentration of each component in the antiviral composition, the aging time and the like.

In addition, the antiviral coating layer is contained as a whole in the thickness direction over the surface and rear surface of the fabric, or an antiviral coating layer is not provided on the surface or rear surface of the fabric, or in the center in the thickness direction of the fabric, and an antiviral coating layer may be treated with the antiviral coating composition so as to be provided only on the surface portion and rear surface portion of the fabric.

Furthermore, when the fabric is composed of multiple layers, any one layer or any two more layers of the multiple layers may be treated with the antiviral coating composition, and through this, the antiviral coating layer may be provided on one layer or any two more layers of the fabric.

The surface of the fabric or yarn constituting the fabric may be treated with the antiviral coating composition through a known coating method, and the known coating method may be, for example, impregnation, spin coating, comma coating, spraying, electrospraying, and the like. In addition, the antiviral coating composition may be implemented to have an appropriate viscosity according to a specific coating method.

When looking specifically at the case where the antiviral coating composition, particularly, the antiviral coating composition further including an aggregation-inducing component, is applied onto the surface of the target by electrospraying, the electrospraying may be performed using a known electrospraying device. In this case, the conditions of the electrospraying are as follows: the distance between a tip and a collector may be 10 to 50 cm, the voltage applied to the tip may be 30 to 70 kV, the temperature during spraying may be 20 to 40° C., the relative humidity may be 30 to 50%, and through this, it may be suitable for implementing an antiviral coating layer which is uniform and exhibits the desired effect of the present invention.

Furthermore, only the surface of the fabric may be treated with the antiviral coating composition, or the surface of the yarn constituting the fabric may be treated with the antiviral coating composition so as to be brought into contact with the antiviral coating composition regardless of the position of the yarn. Further, the antiviral coating layer may be formed by appropriately adjusting the concentration of each component in the antiviral coating layer and aging time, and the like such that the pores retained before treatment are not blocked even after the antiviral coating layer is formed. Alternatively, an antiviral coating layer may be formed such that all the pores of the fabric are blocked depending on the purpose.

In addition, after the surface of the fabric or yarn constituting the fabric is treated with the antiviral coating composition, a reaction may be induced for a predetermined time such that the antiviral coating layer is formed. In this case, the reaction time may vary depending on the concentration of the antiviral fusion protein in the coating composition, the concentration of the aggregation-inducing component, the thickness of the desired coating layer, the time taken for the antiviral coating composition to react after penetrating into the fabric, the temperature, and the like. The reaction may be completed before the drying step after the treatment of the antiviral coating composition, but is not limited thereto, and it should be noted that the reaction may occur after the treatment of the antiviral coating composition, or the reaction may be completed through a drying step, particularly, a drying step performed by applying heat to be described later in a state in which the reaction is only partially completed.

Thereafter, the antiviral coating composition is dried naturally at room temperature for 1 to 24 hours or dried with hot air and/or an IR lamp at a temperature of 30 to 100° C. so that the antiviral fusion protein may form particles on the surface of the target to implement an antiviral coating layer bound to the surface of the target.

Meanwhile, the fabric may contain a silver-containing yarn having antibacterial properties as a yarn. Specifically, the fabric may be implemented so as to have both an antiviral function and an antibacterial function by including a first yarn having an antiviral coating layer to be described below formed thereon and a second yarn containing silver. In this case, the first yarn and the second yarn may constitute a single-layered fabric, or a first fabric layer including the first yarn and having an antiviral function and a second fabric layer including the second yarn and having an antibacterial function may be laminated to constitute a fabric. In addition, when the first yarn and the second yarn constitute a single-layered fabric, the first yarn and the second yarn are each included in the fabric as a single yarn, or the fabric may be implemented through a yarn in which the first yarn and the second yarn are combined. Furthermore, when the first fabric layer and the second fabric layer are laminated to constitute a fabric, for example, the second fabric layer may be a mesh sheet made of a yarn containing silver, more specifically, a mesh sheet composed of silver wires.

In this case, the silver-containing yarn is a yarn in which silver nanoparticles are provided on the surface and/or inside of the yarn, the silver-containing fiber is a silver wire consisting of silver alone, or may be a metal wire containing other metals such as copper other than silver, or a plied yarn formed by combining a silver wire and/or a silver-containing metal wire with a typical non-metallic fiber.

When a metal wire containing other metals such as copper is described, the metal wire may be linearly formed by mixing metals other than silver with silver in a non-solid solution state, that is, with silver in a non-alloyed state. When metals other than silver are mixed with silver in a non-solid solution state, silver and other metals may be disposed such that silver and other metals within a single-stranded linear region regularly or irregularly occupy a predetermined region, respectively, and for example, it may be a double structure in which silver surrounds the outside of a copper wire to form a layer. In this case, the copper wire may impart excellent flexibility to the silver wire, and the surrounding silver may have an average thickness of 3 to 3200 nm, preferably 5 to 3000 nm. When the average thickness of the surrounding silver is less than 3 nm, the antibacterial function may deteriorate because copper, which is the central metal, is likely to prepared so as to be exposed to the outside, the antibacterial function may further deteriorate because silver is desorbed from the silver wire, or there is a concern that desorbed silver may be inhaled into the respiratory tract of a person using silver or may remain on the skin. Further, when the average thickness of the surrounding silver exceeds 3200 nm, the flexibility of the silver wire may deteriorate. Such a double-structured silver wire may be formed by drawing a copper material to a predetermined diameter, integrating the copper material drawn by a cladding process and a silver plate to obtain a double-structured wire in which the silver plate surrounds the outside of the copper material, and obtaining a silver wire by subjecting the double-structured wire to wire drawing processing. Alternatively, the silver wire may be obtained by treating the drawn copper material with a solution containing a liquid Ag powder solution to coat the surface of the copper material with Ag having a uniform thickness, and then performing wire drawing processing. Alternatively, a silver wire may also be obtained by plating the drawn copper material with silver and subjecting the silver-plated drawn copper material to wire drawing processing.

Next, when the form of yarn formed by combining the silver wire with typical non-metallic fibers is described, the silver wire and typical fibers may be a plied yarn implemented by appropriately employing a known manufacturing method in the field of fibers in which two types of fibers are combined and a known arrangement structure of two fibers. In this case, the silver wire used may be a wire consisting only of silver or a metal wire containing silver and other metals. As an example, the plied yarn may be a yarn having a triple-structured cross section, which includes a core yarn, a first covering yarn including a silver wire surrounding the core yarn, and a second covering yarn surrounding the first covering yarn surrounding the core yarn.

The core yarn and the second covering yarn can be used without limitation as long as the core yarn and the second covering yarn are fibers that can be used to improve the flexibility and stretchability of the plied yarn, and preferably, any one selected from a natural fiber and a synthetic fiber may be used, and more preferably, a polyester-based fiber may be used. In addition, the core yarn and the second covering yarn may be formed of mono-filament yarn or a plurality of filament yarns, and may preferably be fibers formed of a plurality of filament yarns. Furthermore, the core yarn and the second covering yarn can be used without limitation as long as the core yarn and the second covering yarn are fibers with a fineness which can be typically used in the art, and preferably, they each may independently have a fineness of 20 to 100 denier (De′), more preferably 30 to 75 De′. When the finenesses of the core yarn and the second covering yarn are each independently less than 20 De′, the washing durability and the ability to maintain the antibacterial performance and washing durability due to the single yarn of the silver wire may deteriorate, and when the fineness exceeds 100 De′, stretchability may deteriorate.

Further, the second covering yarn may be twisted at a twist number of 350 to 1100 TPM, preferably at 450 to 1000 TPM to be included in the plied yarn. When the twist number of the second covering yarn is less than 350 TPM, the washing durability and antibacterial performance due to the single yarn of the silver wire may deteriorate. In addition, when the twist number exceeds 1100 TPM, the stretchability and flexibility of the fabric may deteriorate, and as the area of the silver wire exposed on the surface may be decreased, antibacterial performance may relatively deteriorate.

Furthermore, the present invention may implement various articles using a fabric provided with the above-described antiviral coating layer. The article may be a known fibrous product. Further, the article may be clothing such as a top, a bottom, socks, underwear, gloves, a hat, and earplugs. In addition, the article may be a sanitary product such as various tissues, diapers, sanitary napkins, and waterproof pads. Furthermore, the article may be various types of bedding such as pillowcases, comforters, and flat sheets. Further, the article may be implemented as a filler for various types of clothing, pillows, cushions, mattresses, blankets, and the like, which are composed of fibers or fabrics provided with an antiviral coating layer.

In addition, the present invention may implement various special articles for medical and quarantine uses using a fabric provided with the above-described antiviral coating layer. The article may be a known fibrous product. Furthermore, the article may be, for example, various types of clothing such as surgical gowns, surgical clothing, surgical caps, patient clothing, and quarantine clothing.

The following Table 1 shows amino acid sequences for the above-described antiviral motif and adhesive protein.

TABLE 1

| SEQ ID NO | Amino acid sequence |
|---|---|
| 1 | Lys Lys Lys Lys Tyr Arg Asn Ile Arg Arg Pro Gly |
| 2 | Thr Leu Lys Pro Ile Phe Lys Leu Pro Leu Gly Ile Asn Ile Thr Asn Phe |
| | Arg |
| 3 | Phe His Arg Lys Lys Gly Arg Gly Lys His Lys |
| 4 | Ser Leu Ile Gly Arg Leu |
| 5 | Trp Leu Val Phe Phe Val Ile Phe Tyr Phe Phe Arg Arg Arg Lys Lys |
| 6 | Arg Arg Lys Lys Trp Leu Val Phe Phe Val Ile Phe Tyr Phe Phe Arg |
| 7 | Arg Arg Lys Lys Ala Ala Val Ala Leu Leu Pro Ala Val Leu Leu Ala Leu |
| | Leu Ala Pro |
| 8 | Lys Leu Leu Leu Lys Leu Leu Lys Lys Leu Leu Lys Leu Leu Lys Lys Lys |
| 9 | Ala Lys Pro Ser Tyr Pro Pro Thr Tyr Lys |
| 10 | Ala Lys Pro Ser Tyr Pro Pro Thr Tyr Lys Ala Lys Pro Ser Tyr Pro Pro |
| | Thr Tyr Lys |
| 11 | Ala Lys Pro Ser Tyr Pro Pro Thr Tyr Lys Ala Lys Pro Ser Tyr Pro Pro |
| | Thr Tyr Lys Ala Lys Pro Ser Tyr Pro Pro Thr Tyr Lys Ala Lys Pro Ser |
| | Tyr Pro Pro Thr Tyr Lys Ala Lys Pro Ser Tyr Pro Pro Thr Tyr Lys Ala |
| | Lys Pro Ser Tyr Pro Pro Thr Tyr Lys |
| 12 | Glu Val His Ala Cys Lys Pro Asn Pro Cys Lys Asn Asn Gly Arg Cys Tyr |
| | Pro Asp Gly Lys Thr Gly Tyr Lys Cys Lys Cys Val Gly Gly Tyr Ser Gly |
| | Pro Thr Cys Ala Cys |
| 13 | Ala Asp Tyr Tyr Gly Pro Lys Tyr Gly Pro Pro Arg Arg Tyr Gly Gly Gly |
| | Asn Tyr Asn Arg Tyr Gly Gly Ser Arg Arg Tyr Gly Gly Tyr Lys Gly Trp |
| | Asn Asn Gly Trp Lys Arg Gly Arg Trp Gly Arg Lys Tyr Tyr Glu Phe Glu |
| | Phe |
| 14 | Ala Asp Tyr Tyr Gly Pro Lys Tyr Gly Pro Pro Arg Arg Tyr Gly Gly Gly |
| | Asn Tyr Asn Arg Tyr Gly Arg Arg Tyr Gly Gly Tyr Lys Gly Trp Asn Asn |
| | Gly Trp Lys Arg Gly Arg Trp Gly Arg Lys Tyr Tyr |
| 15 | Gly His Val His Arg His Arg Val Leu His Lys His Val His Asn His Arg |
| | Val Leu His Lys His Leu His Lys His Gin Val Leu His Gly His Val His |
| | Arg His Gin Val Leu His Lys His Val His Asn His Arg Val Leu His Lys |
| | His Leu His Lys His Gin Val Leu His |
| 16 | Ser Ser Glu Glu Tyr Lys Gly Gly Tyr Tyr Pro Gly Asn Ala Tyr His Tyr |
| | His Ser Gly Gly Ser Tyr His Gly Ser Gly Tyr His Gly Gly Tyr Lys Gly |
| | Lys Tyr Tyr Gly Lys Ala Lys Lys Tyr Tyr Tyr Lys Tyr Lys Asn Ser Gly |
| | Lys Tyr Lys Tyr Leu Lys Lys Ala Arg Lys Tyr His Arg Lys Gly Tyr Lys |
| | Lys Tyr Tyr Gly Gly Ser Ser |
| 17 | Ser Ser Glu Glu Tyr Lys Gly Gly Tyr Tyr Pro Gly Asn Thr Tyr His Tyr |
| | His Ser Gly Gly Ser Tyr His Gly Ser Gly Tyr His Gly Gly Tyr Lys Gly |
| | Lys Tyr Tyr Gly Lys Ala Lys Lys Tyr Tyr Tyr Lys Tyr Lys Asn Ser Gly |
| | Lys Tyr Lys Tyr Leu Lys Lys Ala Arg Lys Tyr His Arg Lys Gly Tyr Lys |
| | Lys Tyr Tyr Gly Gly Gly Ser Ser |
| 18 | Tyr Asp Asp Tyr Ser Asp Gly Tyr Tyr Pro Gly Ser Ala Tyr Asn Tyr Pro |
| | Ser Gly Ser His Trp His Gly His Gly Tyr Lys Gly Lys Tyr Tyr Gly Lys |
| | Gly Lys Lys Tyr Tyr Tyr Lys Phe Lys Arg Thr Gly Lys Tyr Lys Tyr Leu |
| | Lys Lys Ala Arg Lys Tyr His Arg Lys Gly Tyr Lys Lys His Tyr Gly Gly |
| | Ser Ser Ser |
| 19 | Ser Ser Glu Glu Tyr Lys Gly Gly Tyr Tyr Pro Gly Asn Thr Tyr His Tyr |
| | His Ser Gly Gly Ser Tyr His Gly Ser Gly Tyr His Gly Gly Tyr Lys Gly |
| | Lys Tyr Tyr Gly Lys Ala Lys Lys Tyr Tyr Tyr Lys Tyr Lys Asn Ser Gly |
| | Lys Tyr Lys Tyr Leu Lys Lys Ala Arg Lys Tyr His Arg Lys Gly Tyr Lys |
| | Lys Tyr Tyr Gly Gly Gly Ser Ser |
| 20 | Gly Gly Gly Asn Tyr Arg Gly Tyr Cys Ser Asn Lys Gly Cys Arg Ser Gly |
| | Tyr Ile Phe Tyr Asp Asn Arg Gly Phe Cys Lys Tyr Gly Ser Ser Ser Tyr |
| | Lys Tyr Asp Cys Gly Asn Tyr Ala Gly Cys Cys Leu Pro Arg Asn Pro Tyr |
| | Gly Arg Val Lys Tyr Tyr Cys Thr Lys Lys Tyr Ser Cys Pro Asp Asp Phe |
| | Tyr Tyr Tyr Asn Asn Lys Gly Tyr Tyr Tyr Tyr Asn Asp Lys Asp Tyr Phe |
| | Asn Cys Gly Ser Tyr Asn Gly Cys Cys Leu Arg Ser Gly Tyr |
| 21 | Ala Lys Pro Ser Tyr Pro Pro Thr Tyr Lys Ala Lys Pro Ser Tyr Pro Pro |
| | Thr Tyr Lys Ala Lys Pro Ser Tyr Pro Pro Thr Tyr Lys Ala Lys Pro Ser |
| | Tyr Pro Pro Thr Tyr Lys Ala Lys Pro Ser Tyr Pro Pro Thr Tyr Lys Ala |
| | Lys Pro Ser Tyr Pro Pro Thr Tyr Lys Ser Ser Glu Glu Tyr Lys Gly Gly |
| | Tyr Tyr Pro Gly Asn Ala Tyr His Tyr His Ser Gly Gly Ser Tyr His Gly |
| | Ser Gly Tyr His Gly Gly Tyr Lys Gly Lys Tyr Tyr Gly Lys Ala Lys Lys |
| | Tyr Tyr Tyr Lys Tyr Lys Asn Ser Gly Lys Tyr Lys Tyr Leu Lys Lys Ala |
| | Arg Lys Tyr His Arg Lys Gly Tyr Lys Tyr Tyr Gly Gly Ser Ser Ala Lys |
| | Pro Ser Tyr Pro Pro Thr Tyr Lys Ala Lys Pro Ser Tyr Pro Pro Thr Tyr |
| | Lys Ala Lys Pro Ser Tyr Pro Pro Thr Tyr Lys Ala Lys Pro Ser Tyr Pro |
| | Pro Thr Tyr Lys Ala Lys Pro Ser Tyr Pro Pro Thr Tyr Lys Ala Lys Pro |
| | Ser Tyr Pro Pro Thr Tyr Lys |
| 22 | Ala Lys Pro Ser Tyr Pro Pro Thr Tyr Lys Ala Lys Pro Ser Tyr Pro Pro |
| | Thr Tyr Lys Ala Lys Pro Ser Tyr Pro Pro Thr Tyr Lys Ala Lys Pro Ser |
| | Tyr Pro Pro Thr Tyr Lys Ala Lys Pro Ser Tyr Pro Pro Thr Tyr Lys Ala |
| | Lys Pro Ser Tyr Pro Pro Thr Tyr Lys Ser Ser Glu Glu Tyr Lys Gly Gly |
| | Tyr Tyr Pro Gly Asn Thr Tyr His Tyr His Ser Gly Gly Ser Tyr His Gly |
| | Ser Gly Tyr His Gly Gly Tyr Lys Gly Lys Tyr Tyr Gly Lys Ala Lys Lys |
| | Tyr Tyr Tyr Lys Tyr Lys Asn Ser Gly Lys Tyr Lys Tyr Leu Lys Lys Ala |
| | Arg Lys Tyr His Arg Lys Gly Tyr Lys Lys Tyr Tyr Gly Gly Gly Ser Ser |
| | Ala Lys Pro Ser Tyr Pro Pro Thr Tyr Lys Ala Lys Pro Ser Tyr Pro Pro |
| | Thr Tyr Lys Ala Lys Pro Ser Tyr Pro Pro Thr Tyr Lys Ala Lys Pro Ser |
| | Tyr Pro Pro Thr Tyr Lys Ala Lys Pro Ser Tyr Pro Pro Thr Tyr Lys Ala |
| | Lys Pro Ser Tyr Pro Pro Thr Tyr Lys |

The present invention will be described in more detail through the following Examples, but the following Examples are not intended to limit the scope of the present invention and should be interpreted to help the understanding of the present invention.

### Example 1

A polyester SDY (75 denier/36 filaments) was woven into a plain woven fabric with a warp density of 156 yarns/inch and a weft density of 102 yarns/inch as a warp yarn and a weft yarn using a Rapier loom weaving machine manufactured by Picanol GTM. The plain woven fabric was scoured (CPB scouring) by a typical method, and then washed with water (B/O), and preset under a condition of 40 m/min at a temperature of 200° C., and then dyed (RAPID, 125° C., 60 min) and subjected to processing (190° C., 40 m/min) to prepare a fabric to be coated.

After a first solution containing an antiviral fusion protein prepared through the following preparation example and water and a second solution in which a carbodiimide-based coupling agent and a hydroxy succinimide-based reactive agent were dissolved in ethanol were introduced into an electrospraying device through separate conduits without being mixed, the device was designed such that the first solution and the second solution entered a spraying pack in the electrospraying device while being mixed through a Y-shaped conduit immediately before the spraying pack, and in this case, the first solution and the second solution were allowed to pass through the Y-shaped conduit, such that the concentration of the antiviral fusion protein in the mixed first solution and second solution was 0.1 mg/ml, and the weight ratio of the total weight of the carbodiimide-based coupling agent and the hydroxy succinimide-based reactive agent and the antiviral fusion protein was 1:1. Thereafter, electrospraying was performed on the surface of the fabric to be coated at a discharge rate of 20 ml/min, a distance of 40 cm between the tip and the collector, at a temperature of 30° C., a relative humidity of 45 RH %, and a voltage of 50 kV applied to the tip.

Thereafter, the fabric was allowed to pass an IR lamp for initial drying, and then dried with hot air at 70° C. to implement an antiviral fabric provided with an antiviral coating layer by forming particles of the antiviral fusion protein immobilized on the surface.

### Preparation Example—Preparation of Antiviral Coating Composition

As the antiviral fusion protein, an antiviral fusion protein was prepared in which the carboxyl group terminus of a mussel-derived adhesive protein which is SEQ ID NO: 21 and the amino terminus of the antiviral motif which is SEQ ID NO: 8 were bound. In this case, the antiviral fusion protein was prepared by a recombinant protein production method using Escherichia coli.

Specifically, the first solution was prepared by dissolving the antiviral fusion protein in water. Further, the second solution was prepared by including a carbodiimide-based coupling agent which is 1-[3-(dimethylamino)propyl]-3-ethylcarboimide hydrochloride (EDC), a reactive agent which is N-hydroxysulfosuccinimide (Sulfo-NHS), sodium acetate which is an active component, and water as a solvent, specifically, including EDC and Sulfo-NHS so as to have a weight ratio of 1:1, including sodium acetate such that the weight ratio of sodium acetate based on the total weight of EDC and Sulfo-NHS was 1:0.3, and then stirring the resulting mixture. Thereafter, the prepared first solution and second solution were stored at 20° C. and 5° C., respectively.

### Example 2

An antiviral fusion protein was prepared in the same manner as in Example 1, except that an antiviral fabric, on which the antiviral fusion protein was immobilized, was prepared by introducing the following antiviral coating composition in which a tyrosine residue of an adhesive protein in the antiviral fusion protein was modified into a DOPA residue into an electrospraying device.

In this case, the modification into a DOPA residue was performed by dissolving the antiviral fusion protein in a buffer solution including 50 mM ascorbic acid to a concentration of 1 mg/ml, and in this case, as the buffer solution, a buffer solution of 40 mM sodium acetate and 20 mM sodium borate was used. Thereafter, the prepared solution was saturated with oxygen while injecting oxygen into the solution for 20 minutes, and then mushroom-derived tyrosinase was added to a final concentration of 35 μg/ml. Thereafter, after mixing and stirring under oxygen conditions for 1 hour, acetic acid was added to a final concentration of 5% to terminate the reaction after the modification reaction into a DOPA residue.

Thereafter, the completed reaction solution was desalted and concentrated with a 5% acetic acid solution, and then subjected to a freeze-drying process to obtain an antiviral fusion protein containing a DOPA residue in a powder form. Thereafter, an antiviral coating composition was prepared by dissolving the obtained antiviral fusion protein to a concentration of 0.1 mg/ml using a 40 mM Tris buffer with a pH of 8.2.

### Example 3

An antiviral fabric was prepared in the same manner as in Example 1, except that an antiviral fabric, in which the antiviral fusion protein was provided, was prepared using a solution of the antiviral fusion protein dissolved in water as an antiviral coating composition.

### Comparative Example 1

An antiviral fabric was prepared in the same manner as in Example 1, except that an antiviral fabric, in which an antiviral motif was provided, was prepared by changing the antiviral fusion protein into the antiviral motif alone.

### Experimental Example 1

The following physical properties were examined for the antiviral fabrics having surfaces provided with the antiviral coating layer prepared in Examples 1 to 3 and Comparative Example 1, and the results are shown in the following Table 2.

1. Antiviral Performance

An antiviral fabric on which an antiviral coating layer was formed was prepared so as to have a width of 4 cm and a length of 4 cm. Thereafter, after each sample was treated with 200 ml of PED virus (coronaviridae, enveloped RNA virus) and then allowed to stand at 23° C. for 14 hours, the virus located on the sample was collected by adding 800 μl of an inoculation medium which is 1×DMEM (0.3% tryptose phosphate broth, 0.02% yeast extract, 1% antibiotic-antimycotic, 5 μg/ml trypsin) to the sample.

After an inoculation medium containing the collected virus was diluted by decimal dilution, 100 μl of the inoculation medium was inoculated into 5 wells per dilution factor, then adsorbed for 1 hour in a CO₂ incubator, then the inoculum was removed, and 200 μl of a virus culture medium per well was aliquoted to a 96-well plate containing VERO cells at 200×10⁴ cells/well and incubated in an incubator for 5 days. Thereafter, the CPE of the cells was confirmed and the TCID value was calculated, and the resulting values are shown in the following Table 2.

The TCID log conversion value of the viral titer was 5.0000, a positive control was a result evaluated by allowing only 200 μl of the viral stock solution to stand at a temperature of 23° C. for 14 hours, and the TCID log conversion value was 5.0000.

2. Adhesion Performance

For Examples 1 to 3 which had antiviral performance among the antiviral fabrics with an antiviral surface, impregnating each sample in water at 23° C. and then taking out the sample after 1 minute was defined as one set, and 20 sets were performed, and then the above antiviral performance was evaluated.

3. Storage Stability

After an accelerated aging test according to guidelines for setting the shelf life of medical devices and evaluating stability was performed by the following method, the storage stability of the fabric with an antiviral surface was evaluated by evaluating the above-described antiviral performance.

Specifically, in order to reproduce the real-time aging of the antiviral fabric within a shortened period of time, the porous substrate was prepared such that the aging period of each antiviral fabric was 3 years by storing the porous substrate at an elevated temperature (60° C.) for 3 months.

TABLE 2

| | | | | Comparative |
|---|---|---|---|---|
| | Example 1 | Example 2 | Example 3 | Example 1 |
| Immobilization | Antiviral | Antiviral fusion | Antiviral | Antiviral motif |
| target | fusion protein | protein | fusion protein | |
| Additional | EDC + Sulfo- | Antiviral fusion | None | None |
| immobilization | NHS | protein DOPA | | |
| method | | modification | | |
| Antiviral | 3.6 | 3.8 | 4.5 | 5.0 |
| performance | | | | |
| (TCID log | | | | |
| conversion | | | | |
| value) | | | | |
| Adhesion | 3.6 | 4.0 | 5.0 | Not measured |
| performance | | | | |
| (TCID log | | | | |
| conversion | | | | |
| value) | | | | |
| Storage | 3.8 | 4.7 | 5.0 | Not measured |
| stability (TCID | | | | |
| log conversion | | | | |
| value) | | | | |

As can be confirmed from Table 2, it can be seen that the antiviral performance of the surfaces having the antiviral coating layers formed according to Examples 1 and 2 is effectively present at 90% or more.

In addition, as a result of evaluating adhesion performance, there was little change in the antiviral performance of Examples 1 and 2, and in particular, there was no change in the antiviral performance according to Example 1, and through this, it can be seen that the method of immobilizing an antiviral fusion protein through Example 1 exhibits excellent adhesion characteristics on the surface.

Furthermore, as a result of the storage stability evaluation, Example 1 showed significantly less reduction in antiviral performance than the other Examples, and through this, it can be seen that the storage stability and the sustainability of the antiviral effect are excellent.

Although one embodiment of the present invention has been described above, the spirit of the present invention is not limited to the embodiments presented in the present specification, and a person skilled in the art who understands the spirit of the present invention can easily propose other embodiments by substitutions, changes, deletions, additions, and the like of the constituent elements, but it can be said that those embodiments also fall within the scope of the spirit of the present invention.

1. An antiviral fabric comprising: a fabric; and
an antiviral coating layer provided on the fabric and comprising an antiviral fusion protein in which an antiviral motif is bound to an adhesive protein.

2. The antiviral fabric of claim 1, wherein the antiviral motif targets a protein that binds to a host cell receptor to disable or disrupt the protein or perform a function of disrupting the viral membrane.

3. The antiviral fabric of claim 1, wherein the adhesive protein is a mussel-derived adhesive protein.

4. The antiviral fabric of claim 1, wherein the antiviral motif is any one peptide selected from the group consisting of amino acid sequences of SEQ ID NOS: 1 to 8 or a peptide in which one or more amino acid sequences selected from the above group are linked, and
the adhesive protein is any one protein selected from the group consisting of amino acid sequences of SEQ ID NOS: 9 to 22 or a protein in which one or more amino acid sequences selected from the above group are linked.

5. The antiviral fabric of claim 1, wherein the antiviral coating layer is formed on fabrics by aggregating particles formed of the antiviral fusion protein.

6. The antiviral fabric of claim 5, wherein the antiviral coating layer is formed through an antiviral coating composition comprising an antiviral fusion protein and an aggregation-inducing component comprising a carbodiimide-based coupling agent and a hydroxy succinimide-based reactive agent.

7. The antiviral fabric of claim 1, wherein the adhesive protein contains a DOPA residue, and the antiviral fusion protein is immobilized on a fiber through the DOPA residue.

8. The antiviral fabric of claim 7, wherein the DOPA residue is a DOPA residue into which some or all tyrosine residues of the adhesive protein are modified through an enzyme.

9. The antiviral fabric of claim 1, wherein the fabric comprises any one or more of a woven fabric, a knitted fabric and a non-woven fabric.

10. The antiviral fabric of claim 1, wherein the fabric is composed of a single layer such that at least a part thereof has a moisture-permeable waterproof function, or may be composed of multiple layers including at least one moisture-permeable waterproof layer.

11. The antiviral fabric of claim 10, wherein the moisture-permeable waterproof layer is a nanofiber web provided with a fiber-forming component comprising a fluorine-based compound and any one or more additives selected from a water repellent, an oil repellent, and a moisture absorbent.

12. The antiviral fabric of claim 1, wherein the fabric comprises a first yarn having an antiviral coating layer formed on the surface and a second yarn containing silver.

13. The antiviral fabric of claim 12, wherein the first yarn and the second yarn constitute a single-layered fabric, or
a first fabric layer comprising the first yarn and a second fabric layer comprising the second yarn are laminated to constitute a fabric.

14. A sanitary product comprising the antiviral fabric according to claim 1.

15. An article for medical and quarantine uses, comprising the antiviral fabric according to claim 1.


SEQUENCE LISTING (22 sequences)

SEQ ID NO: 1 - protein, 12 aa, Artificial Sequence
  Antiviral peptide
KKKKYRNIRRPG

SEQ ID NO: 2 - protein, 18 aa, Artificial Sequence
  Antiviral peptide
TLKPIFKLPLGINITNFR

SEQ ID NO: 3 - protein, 10 aa, Artificial Sequence
  Antiviral peptide
AKPSYPPTYK

SEQ ID NO: 4 - protein, 6 aa, Artificial Sequence
  Antiviral peptide
SLIGRL

SEQ ID NO: 5 - protein, 16 aa, Artificial Sequence
  Antiviral peptide
WLVFFVIFYFFRRRKK

SEQ ID NO: 6 - protein, 48 aa, Artificial Sequence
  Antiviral peptide
ARGARGLYSLYSTRPLEAVALPHEPHEVALILEPHETYRPHEPHEARG

SEQ ID NO: 7 - protein, 20 aa, Artificial Sequence
  Antiviral peptide
RRKKAAVALLPAVLLALLAP

SEQ ID NO: 8 - protein, 17 aa, Artificial Sequence
  Anti-viral motif
KLLLKLLKKLLKLLKKK

SEQ ID NO: 9 - protein, 10 aa, Artificial Sequence
  adhesive protein
AKPSYPPTYK

SEQ ID NO: 10 - protein, 20 aa, Artificial Sequence
  adhesive protein
AKPSYPPTYKAKPSYPPTYK

SEQ ID NO: 11 - protein, 60 aa, Artificial Sequence
  adhesive protein
AKPSYPPTYKAKPSYPPTYKAKPSYPPTYKAKPSYPPTYKAKPSYPPTYKAKPSYPPTYK

SEQ ID NO: 12 - protein, 39 aa, Artificial Sequence
  adhesive protein
EVHACKPNPCKNNGRCYPDGKTGYKCKCVGGYSGPTCAC

SEQ ID NO: 13 - protein, 52 aa, Artificial Sequence
  adhesive protein
ADYYGPKYGPPRRYGGGNYNRYGGSRRYGGYKGWNNGWKRGRWGRKYYEFEF

SEQ ID NO: 14 - protein, 46 aa, Artificial Sequence
  adhesive protein
ADYYGPKYGPPRRYGGGNYNRYGRRYGGYKGWNNGWKRGRWGRKYY

SEQ ID NO: 15 - protein, 60 aa, Artificial Sequence
  adhesive protein
GHVHRHRVLHKHVHNHRVLHKHLHKHQVLHGHVHRHQVLHKHVHNHRVLHKHLHKHQVLH

SEQ ID NO: 16 - protein, 75 aa, Artificial Sequence
  adhesive protein
SSEEYKGGYYPGNAYHYHSGGSYHGSGYHGGYKGKYYGKAKKYYYKYKNSGKYKYLKKARKYHRKGYKKYYGGSS

SEQ ID NO: 17 - protein, 76 aa, Artificial Sequence
  adhesive protein
SSEEYKGGYYPGNTYHYHSGGSYHGSGYHGGYKGKYYGKAKKYYYKYKNSGKYKYLKKARKYHRKGYKKYYGGGSS

SEQ ID NO: 18 - protein, 71 aa, Artificial Sequence
  adhesive protein
YDDYSDGYYPGSAYNYPSGSHWHGHGYKGKYYGKGKKYYYKFKRTGKYKYLKKARKYHRKGYKKHYGGSSS

SEQ ID NO: 19 - protein, 76 aa, Artificial Sequence; identical to SEQ ID NO: 17
  adhesive protein

SEQ ID NO: 20 - protein, 99 aa, Artificial Sequence
  adhesive protein
GGGNYRGYCSNKGCRSGYIFYDNRGFCKYGSSSYKYDCGNYAGCCLPRNPYGRVKYYCTKKYSCPDDFYYYNNKGYYYYNDKDYFNCGSYNGCCLRSGY

SEQ ID NO: 21 - protein, 194 aa, Artificial Sequence
  adhesive protein
AKPSYPPTYKAKPSYPPTYKAKPSYPPTYKAKPSYPPTYKAKPSYPPTYKAKPSYPPTYKSSEEYKGGYYPGNAYHYHSGGSYHGSGYHGGYKGKYYGKAKKYYYKYKNSGKYKYLKKARKYHRKGYKYYGGSSAKPSYPPTYKAKPSYPPTYKAKPSYPPTYKAKPSYPPTYKAKPSYPPTYKAKPSYPPTYK

SEQ ID NO: 22 - protein, 194 aa, Artificial Sequence; identical to SEQ ID NO: 21
  adhesive protein